(19) United States
(12) Patent Application Publication
(10) Pub. No.: US 2011/0268612 A1
(43) Pub. Date: Nov. 3, 2011

(54) MICROCHANNEL INTO WHICH A POROUS POLYMER IS INSERTED

(76) Inventor: Dae-Sung Hur, Seoul (KR)

(21) Appl. No.: 13/142,317

(22) PCT Filed: Dec. 21, 2009
(86) PCT No.: PCT/KR2009/007632

§ 371 Date: Jun. 27, 2011

(30) Foreign Application Priority Data
Dec. 29, 2008 (KR) 10-2008-0135861

(51) Int. Cl. G01N 33/00; B01J 8/02

(52) U.S. Cl. 422/68.1; 422/82.01; 422/82.05; 422/211

(57) ABSTRACT

A microchannel is disclosed. The present invention relates to a microchannel having a porous polymer inserted therein, more particularly, to a microchannel including a channel body and a porous polymer inserted in a surface of the channel body to be employed as a valve which can locally maximize a surface area of reaction between samples and reagents and which can control flow of a fluid in the microchannel.

### TECHNICAL FIELD

The present invention relates to a microchannel having a porous polymer inserted therein, more particularly, to a microchannel including a channel body and a porous polymer inserted in a surface of the channel body to be employed as a valve which can locally maximize a surface area of reaction between samples and reagents and which can control flow of a fluid in the microchannel.

### BACKGROUND ART

Analysis of fluid samples has been broadly used not only in chemistry and biotechnology but also in diagnostics based on analysis of blood and body fluid taken from patients and the like. In recent, a variety of compact-sized analysis and diagnosis equipments and technologies haven been under development to perform the analysis of the fluid samples more simply and more efficiently.

Especially, lab-on-a-chip technology refers to technology which can represent on a micro-sized chip a variety of experimental processes including separation, mixture, labeling, analysis and cleaning with respect to samples which are performed in a laboratory, using microfluidics. There has been developing a portable DNA analysis device for personal identification which can implement a process from DNA extraction to DNA analysis on a chip at one time and the lab-on-a-chip technology has been utilized actively in various industries.

In addition, in vitro diagnostics, there have been active studies and researches on a portable diagnosis tool, in other words, Point of Care Testing (POCT) which is used by an individual directly and easily to implement complex throughout medical examinations including blood tests, body fluid tests in a hospital or laboratory on the spot.

The POCT refers to presence diagnosis technology which can conveniently diagnose a disease in the act of medical treatment at an emergency room, an operation room or a household. Demands and necessity for the POCT devices have been increasing more and more for an aging and welfare society. At this present, a diagnostic tool for measuring blood glucose has been central mainstream market. As substantial demands for the POCT are increasing, demands for diagnostic tools capable of analyzing lactic acid, cholesterol, urea, infectious pathogens and various biomaterials have been increasing rapidly.

According to such the analysis or diagnosis technologies, reaction between fluid and an antibody protein or various samples immobilized in the chip is detected and analyzed based on various detecting methods, while various fluid samples are moved via a microchannel formed in a chip.

However, to gain a rapid and accurate analysis result, the movement of the fluid in the chip where the microchannel is formed has to be controlled, proper to a purpose, and the reaction between the analysis sample and the reagent immobilized in the chip has to be performed enough to analyze an object material, with a small quantity of the analysis samples.

To solve those disadvantages, there have been studies on microchannels, for example, a microchannel having different surface characters including hydrophilic and hydrophobic characters which are used to adjust flow of the fluid samples (WO99/058245, 1999 Nov. 18) or a microchannel including a series of electrodes used to adjust flow of the fluid in the microchannel (WO08/052,363, 2008 May 8). Those studies have a disadvantage of a complex fabrication process or a disadvantage of additional devices which have to be attached, rather than a reaction part.

Moreover, to maximize the reaction between the fluid and the reagent, a surface area of the reaction is maximized by using various structures including a membrane, a pillar and a pattern in the microchannel. However, those structures used to maximize the surface area of the reaction might interfere with the flow of the fluid and make it difficult to adjust the flow.

Because of that, a microchannel is required which can maximize the surface area of the reaction between the fluid and the reagent, without interfering with the flow of the fluid at all, and which can adjust the flow properly, if necessary.

In addition, as demands for simultaneous performance of various analyses and integration of various functions have been increasing, demands have been increasing for a multi-functional microchannel which can perform analysis using various reagents on a single chip if necessary and which can perform auxiliary addition of the reagent and immobilization of the reagent smoothly and which can perform filtering and chromatography of the fluid sample simultaneously.

As a result, the present inventor invents a multi-functional microchannel which can control the flow of the fluid to make the reaction time be sufficient, with maximizing the reactivity, and which can perform analysis and detection using various reagents via a single microchannel simultaneously, and which can localize a region where reaction occurs, with an effect of auxiliary filtering and chromatography.

### DISCLOSURE OF INVENTION

### Technical Problem

An object of the present invention is to provide a microchannel which can create a delaying effect of delaying and controlling fluid flow to maximize reaction efficiency and to generate sufficient reaction, without interfering with the flow of the fluid, by maximizing a surface area of reaction between fluid and reagents.

Another object of the present invention is to provide a microchannel which can perform analysis and detection on a single chip simultaneously by using various reagents and which can localize regions where the reaction occurs and which can perform addition and immobilization of reagents easily.

A further object of the present invention is to provide a multifunctional microchannel which can perform filtering and chromatography of fluid reagent simultaneously and which can be applicable to a variety of fields including lab-on-a-chip (LOC) and Point of Care (POC) fields.

### Technical Solution

To achieve these objects and other advantages and in accordance with the purpose of the invention, as embodied and broadly described herein, a microchannel includes a channel body; and a porous polymer inserted in a surface of the channel body to be employed as a valve configured to locally maximize a reaction surface area between an analysis sample and a reagent and to adjust fluid flow in the microchannel.

A reagent hole may be formed in the channel body to add the reagent to the porous polymer.

### Advantageous Effects

The present invention has following advantageous effects. The microchannel can create a delaying effect of delaying and controlling fluid flow to maximize reaction efficiency and to generate sufficient reaction, without interfering with the flow of the fluid, by maximizing a surface area of reaction between fluid and reagents.

Furthermore, the microchannel according to the present invention can perform analysis and detection on a single chip simultaneously by using various reagents. It can localize regions where the reaction occurs and perform addition and immobilization of reagents easily.

Still further, the microchannel according to the present invention can perform filtering and chromatography of fluid reagent simultaneously. The microchannel has multi-functions and it can be applicable to a variety of fields including lab-on-a-chip (LOC) and Point of Care (POC) fields.

### BRIEF DESCRIPTION OF THE DRAWINGS

The accompanying drawings, which are included to provide further understanding of the disclosure and are incorporated in and constitute a part of this application, illustrate embodiments of the disclosure and together with the description serve to explain the principle of the disclosure.

In the drawings:

FIG. 1 is a conceptual diagram illustrating a microchannel having a polymer inserted therein according to the present invention;

FIG. 2 is a chip for fluid analysis according to an embodiment which the microchannel is used;

FIG. 3 is a diagram illustrating porous polymers to which a series of antibodies are attached after a blood sample is injected into the microchannel according to the present invention; and

FIG. 4 is a diagram illustrating porous polymers to which a series of antibodies are attached after a blood sample is injected into the microchannel according to the present invention.

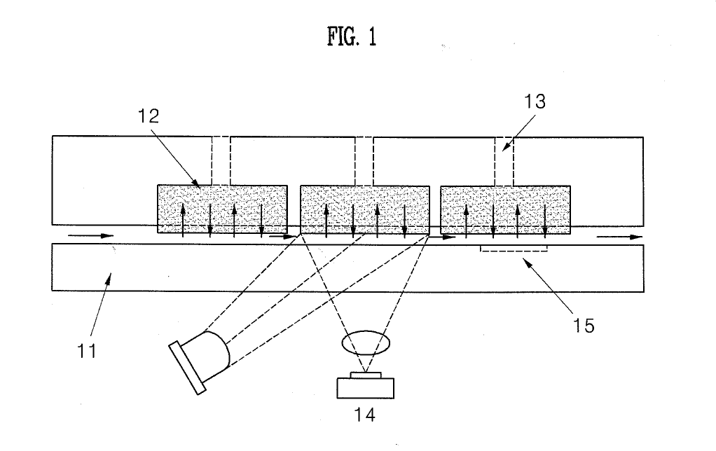

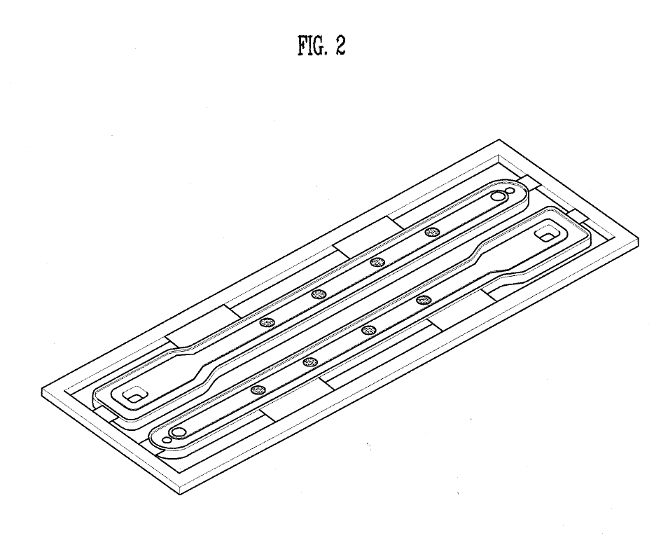

### BEST MODE

As follows, a microchannel according to the present invention will be described in detail in reference to the accompanying drawings.

The microchannel according to the present invention includes a channel body 11 and a porous polymer 12 inserted in a surface of the channel body 11 to be used as a valve capable of maximizing a reaction surface area between an analysis sample and a chemical reagent locally and of adjusting flow of fluid in the microchannel.

When it is injected into the microchannel to pass the porous polymer inserted in the channel body, the analysis sample is absorbed into the porous polymer upwardly and the upwardly absorbed analysis sample may react with a reaction reagent.

At this time, a broad surface area of the porous polymer may maximize reaction efficiency between the analysis sample and the reaction reagent and may localize a reaction region to generate the reaction only at a desired region. In other words, the microchannel according to the present invention may perform more accurate analysis with a much less quantity of samples, compared with a conventional strip type microchannel.

Furthermore, the insertion height of the porous polymer with respect to the channel body may be adjusted appropriately not to interfere with the flow of the sample passing the channel. While the sample is absorbed into and getting out of the porous polymer, fluid flow may be delayed (hereinafter, a delaying effect). Because of that, the porous polymer can be functioned to control the flow of the analysis sample in the channel such that all reactions of the sample may occur sufficiently.

Still further, a surface of the porous polymer is treated to have a special character, for example, a hydrophilic or hydrophobic character. Because of that, additional effects may be achieved. For example, the flow can be controlled or the reagent may be fixed stably. Compared with direct treatment performed to a surface of the microchannel to allow the surface to have such the characters, this treatment according to the present invention mentioned above may be more efficient in the fabrication process and in treating only the reacting region selectively.

In other words, a solvent or a blocking agent can be used to adjust the surface area of the porous polymer within the channel. If necessary, the blocking agent is used to control reaction to occur only at the surface of the porous polymer.

A proper porous polymer may be selected according to the analysis sample and the reaction reagent. Because of that, filtering and chromatography effects with respect to a liquid sample which will be analyzed may be additionally achieved. At this time, the porous polymer may be formed of any materials which can be used and it is preferable that melting point a material used to form the porous polymer is similar with or higher than a melting point of a material used to form the microchannel, in an insert molding process.

A reagent hole 13 may be formed in the channel body 11 to add the reagent to the porous polymer. An experimenter adds the reagent via the reagent hole to perform additional reaction or serial or phased reaction procedure. Also, the experimenter may introduce the solvent or the blocking agent via the reagent hole and the reagent may be fixed easily.

The plurality of the porous polymers may be formed in a single microchannel. Because of that, various analyses may be performed by using the single microchannel and a fabrication cost may be reduced. In addition, various results may be compared with each other to be analyzed.

Various materials may be used to form the channel body 11, for example, transparent, translucent and opaque materials. However, to identify reaction between the analysis sample and the reagent which occurs in the porous polymer through the naked eye easily, it is preferable that the channel body 11 is formed of a transparent or translucent material.

The reagent reaction generated in the porous polymer may be detected through various methods. It may be identified through the naked eye or various detection devices may be applied to detect the reagent reaction, based on a type of reaction. The detection devices may include an optical sensor 14 which sense variation of an optical signal in the porous polymer 12 and an electrochemical sensor which sense variation an electrochemical signal.

The microchannel including the porous polymer may be manufactured of a plastic material in an insert molding process and it can be manufactured smoothly and efficiently. In addition, the microchannel including the porous polymer described above may be applicable to a variety of fields including lab-on-a-chip (LOC) and Point of Care (POC) fields.

As follows, an embodiment of the microchannel according to the present invention which is used to measure a blood type will be described in detail.

### Embodiment

### Blood Type Measurement by Using the Microchannel According to the Present Invention

The microchannel having the porous polymer inserted therein is insert-molded and it is used as top plate. The porous polymer may be poly ethylene (PE) and resin injection is used to fabricate the channel.

A surface of the channel is modified to be hydrophilic by using vacuum plasma. After that, A, B and D antibodies used in ABO typing are fixed to the porous polymer according to a lyophilization method. The top plate having the antibodies fixed there to is adhered to a bottom plate, such that a plastic chip having the porous polymer may be fabricated (see FIG. 3)

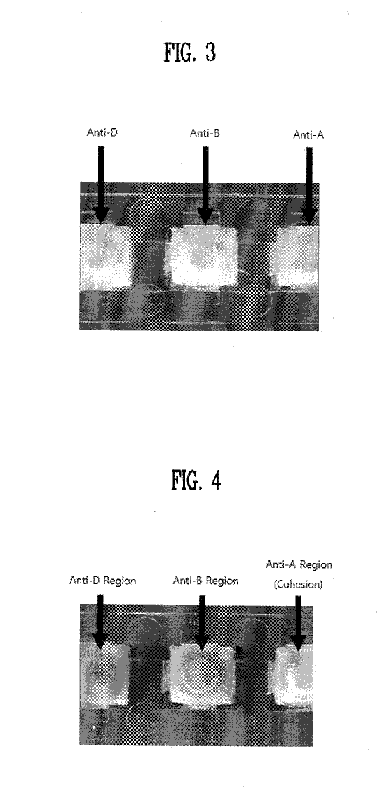

A type blood is injected into the fabricated chip and the blood is flowing, with wetting the channel sequentially. The blood flowing in the channel may react with the A, B and D antibodies fixed to the porous polymer sequentially. The blood penetrates into micro-pores of Anti-B and anti-D regions not reacting with the antibodies to look red through the naked eye, without reaction between the blood and the reagent fixed to the porous polymer. In contrast, the blood reacts with the reagent fixed to the porous polymer at an anti-A region and cohesion occurs on the surface of the porous polymer to allow the blood not to flow into the micro-pores, to generate little color variation (see FIG. 4).

As appears by the embodiment described above, the reaction between the sample and the reagent may be induced sequentially by using the microchannel according to the present invention. The staying time of the sample may be adjusted for each of the reaction regions by adjusting the porous polymer. In addition, simultaneous reaction may be induced according to channel types.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A microchannel comprising:
a channel body; and
a porous polymer inserted in a surface of the channel body to be employed as a valve configured to maximize a reaction surface area between an analysis sample and a reagent locally and to adjust fluid flow in the microchannel.

2. The microchannel as claimed in claim 1, wherein a reagent hole is formed in the channel body to add the reagent to the porous polymer.

3. The microchannel as claimed in claim 1, wherein the plurality of the porous polymers inserted in the channel body are provided.

4. The microchannel as claimed in claim 1, wherein the channel body is formed of a transparent or translucent material to identify the reaction between the analysis sample and the reagent in the porous polymer through the naked eye.

5. The microchannel as claimed in claim 1, further comprising:
a sensor which is able to detect the reaction between the analysis sample and the reagent in the porous polymer.

6. The microchannel as claimed in claim 5, wherein the sensor may be an optical sensor which is able to detect variation of an optical signal in the porous polymer.

7. The microchannel as claimed in claim 5, wherein the sensor may be an electrochemical sensor which is able to detect variation of an electrochemical signal in the porous polymer.

8. The microchannel as claimed in claim 2, wherein the plurality of the porous polymers inserted in the channel body are provided.

9. The microchannel as claimed in claim 2, wherein the channel body is formed of a transparent or translucent material to identify the reaction between the analysis sample and the reagent in the porous polymer through the naked eye.

10. The microchannel as claimed in claim 2, further comprising:
a sensor which is able to detect the reaction between the analysis sample and the reagent in the porous polymer.
